(19) United States
(12) Patent Application Publication
(10) Pub. No.: US 2020/0368294 A1
(43) Pub. Date: Nov. 26, 2020

(54) THICKENING COMPOSITION FOR DYSPHAGIA PATIENTS

(71) Applicant: MITSUBISHI CORPORATION LIFE SCIENCES LIMITED, Tokyo (JP)

(72) Inventors: Yusuke MATSUYAMA, Tokyo (JP); Ryo KAGOTANI, Tokyo (JP); Maki IMAOKA, Tokyo (JP); Atsushi KAMURA, Tokyo (JP)

(73) Assignee: Mitsubishi Corporation Life Sciences Limited, Tokyo (JP)

(21) Appl. No.: 16/634,245

(22) PCT Filed: Jul. 26, 2018
(86) PCT No.: PCT/JP2018/028017

§ 371 Date: Jan. 27, 2020

(30) Foreign Application Priority Data
Jul. 31, 2017 (JP) 2017-147942

(51) Int. Cl. A61K 35/741; A61K 9/16; A61K 9/00; A23L 33/135; A23L 33/125; A23P 10/20

(52) U.S. Cl. CPC A61K 35/741; A61K 9/1652; A61K 9/0095; A23V 2002/00; A23L 33/125; A23P 10/20; A23L 33/135

(57) ABSTRACT

An object of the present invention is to provide, when preparing food products for dysphagia patients, a food product for dysphagia patients having useful functions of probiotics and capable of delivering probiotics administered to a dysphagia patient to the intestines as viable cells by imparting functions of probiotics to such food products, and a thickening composition therefor. For achieving these objects, the present invention provides a thickening composition for dysphagia patients prepared as a powder mixture by mixing dried probiotic cells or a dried probiotic cell preparation and one or more polysaccharides selected from the group consisting of polysaccharides consisting of xanthan gum or guar gum and hydrolysates of these polysaccharides, wherein in the powder mixture, the probiotics and the polysaccharides are allowed to coexist in a contact state. The thickening composition for dysphagia patients is prepared as a powder mixture and the probiotics in the thickening composition can be stably retained without employing special conditions, and when a food product for dysphagia patients is produced by using the thickening composition, a probiotics-containing food product for dysphagia patients having stability against decomposition action by gastric juice after such a food product is eaten can be provided.

### TECHNICAL FIELD

The present invention relates to, when preparing food products for dysphagia patients, a thickening composition used for viscosity adjustment of such food products, and relates to a provision of a thickening composition capable of imparting useful functions of probiotics to the food products for dysphagia patients by imparting functions of probiotics to the thickening composition and using such a thickening composition. Further, the thickening composition for dysphagia patients has the assured stability of probiotics contained in the thickening composition and the stability of probiotics against gastric juice decomposition action when a food product for dysphagia patients prepared using the thickening composition for dysphagia patients is eaten, and the present invention relates to a provision of a thickening composition for dysphagia patients having good stability and functionality capable of stably imparting useful functions of probiotics to dysphagia patients via food products for dysphagia patients by using the thickening composition for dysphagia patients. The present application claims the priority of Japanese Patent Application No. 2017-147942 filed on Jul. 31, 2017 and the contents thereof are incorporated herein by reference.

### BACKGROUND ART

With the progress of population aging, a demand is increasing for thickening compositions, so-called a texture modifier, used for the purpose of adjusting a viscosity of foods and drinks for dysphagia patients. Currently, a base agent used for texture modifiers is xanthan gum, but xanthan gum poses the problems of having low dispersibility and easily forming “lumps.” Thus, various arrangements have been proposed to solve these problems. For example, disclosed includes an arrangement wherein xanthan gum and a water-soluble dispersing agent are granulated respectively to achieve low adhesiveness and high agglutination (Patent Document 1), an arrangement wherein a potassium salt solution is sprayed on the xanthan gum surface and then fluid dried (Patent Document 2), an arrangement wherein alginate and CMC are bound onto the xanthan gum surface to prevent lumps when used (Patent Document 3), an arrangement wherein a solution of a metal salt such as a sodium salt, a potassium salt, or a magnesium salt is sprayed onto the powder surface of xanthan gum and then fluid dried to prevent the formation of lumps (Patent Document 4), an arrangement wherein a granular is produced using calcium chloride, calcium lactate, or calcium pantothenate as a water-soluble binder (Patent Document 5), an arrangement wherein dextrin is added to a polysaccharide thickener such as xanthan gum or guar gum to reduce lump formation (Patent Document 6), and an arrangement wherein dextrin having a DE value of 5 or less is added to a water-soluble polymer such as xanthan gum or carrageenan and the mixture is granulated to prevent lumping (undissolved lumps, lump formation) of the water-soluble polymer (Patent Document 7).

Further, xanthan gum is easily thickened with water and can express a predetermined physical property (viscosity) in a short time but poses the problem of extremely delayed viscosity expression to food products and drink products containing a comparatively large number of ions such as a miso soup, a mineral balance drink, a cow's milk, and an orange juice. In response to this problem, disclosed includes a method for adjusting the particle size of xanthan gum (Patent Document 8), a method for spraying a potassium solution onto the xanthan gum powder surface (Patent Document 2), and a method for dissolving xanthan gum in water and then vacuum drying or vacuum freeze drying the solution (Patent Document 9). As described, xanthan gum, although having the problems when used for viscosity adjustment of a thickening composition, has been quite commonly used as the mainstream base agent for texture modifiers due to the development and proposals for solving these problems.

On the other hand, “probiotics” defined in 1989 by Fuller, a British microbiologist, as “living microorganisms which render beneficial actions to people by improving gut flora balance” are reported to have many actions such as regulation of intestinal functions, maintenance and improvement of gut flora, growth reduction of harmful bacteria and viruses, immunostimulatory action, and production reduction of carcinogenic substances and have been widely utilized in pharmaceutical products and food products. When probiotics are eaten in a form of food products to render useful functions of probiotics, there is an obstacle such as decomposition action by gastric juice before the probiotics eaten reach the inside of the intestines. Arrangements in response to such a problem have been also studied. For example, in the probiotic preparations, there is a disclosure to provide a probiotic preparation stabilized against decomposition action of gastric juice by containing, as matrix components, dry-stabilized microorganisms (probiotics) and a gastric juice decomposition resistant substance consisting of sodium alginate, hydroxypropyl methylcellulose, and derivatives thereof, methacrylic acid derivatives and galactomannan, and mixtures thereof (Patent Document 10).

Probiotics, as described above, are reported to have many useful actions and effective utilization thereof is expected to be beneficial for not only healthy person but also people with various disorders. As one of them, such an effective utilization is also expected among dysphagia patients who are affected in the intestinal function due to the dysphagia disorder. For example, dysphagia patients often have an insufficient water intake, and for this reason when the functions of probiotics as described above can be utilized to prepare food products for dysphagia patients to manage the health of dysphagia patients, the effects thereof are notable. Further, in addition to such effects, there is also a notable advantage for dysphagia patients to eat probiotics which are expected to improve constipation.

An attempt in adding probiotics as one of the nutrient components when preparing food products for dysphagia patients has been made. For example, disclosed is a nutritional product for dysphagia patients which is prepared as a nutritional product containing a biopolymer aqueous solution for food products such as microbial hydrocolloids including xanthan gum, gellan gum, and curdlan capable of imparting relaxation time of more than 10 ms (millisecond) at a shear viscosity of about less than 100 mPas and a temperature of 20° C. and improved in such a way as to facilitate safe swallowing for dysphagia patients, in which probiotics are contained as one of the nutrient components for such a nutritional product (Patent Document 11). Further, disclosed is a thickener-containing compound for dysphagia patients prepared by adding 0.03 to 0.05% by weight of carrageenan, 1.5 to 4.0% by weight of starch, and nutrients and adjusted to a viscosity of such a nutrients-containing compound to 300 to 9000 cps thereby to minimize the increase in viscosity of the nutrient compound in food products for dysphagia patients at a refrigerated storage temperature, and is improved in such a way as to provide a consistent viscosity of the nutrient composition during a long storage period and at different temperatures in which probiotics is added as one of the components added as nutrient components such as a carbohydrate, a protein, a fat, a phytonutrient, a vitamin, and a mineral (Patent Document 12, Patent Document 13).

These nutritional products for dysphagia patients and thickener-containing compounds for dysphagia patients are described as probiotics being added thereto as one of the compounds, however, the stability of probiotics, that is, the stability of probiotics in products containing probiotics, and the stability of probiotics against gastric juice decomposition action which will be a problem after such nutritional products and compounds are eaten are not particularly considered; and further these nutritional products and compounds are prepared as viscous food products for dysphagia patients thus requiring, when utilized, an arrangement such as refrigerated storage as described above whereby restrictions on effectiveness and handling remain as the food products for dysphagia patients containing probiotics. Particularly, a food product for dysphagia patients, when utilized, employs a form of being prepared and eaten at the time of eating by adding each component, but those disclosed above cannot be arranged for such a form.

As described above, it is expected to impart functions of probiotics to food products for dysphagia patients and used to manage the health of dysphagia patients utilizing useful functions of probiotics by eating the food products; however, an effective food product for dysphagia patients applicable to various food products for dysphagia patients and capable of delivering useful functions of probiotics to the intestines while administered probiotics remain as viable cells via a food product for dysphagia patients and stably imparting to dysphagia patients has not been provided so far, whereby the development of food products satisfying such a need has been a challenge in the food products for dysphagia patients.

### PRIOR ART DOCUMENTS

### Patent Documents

Patent Document 1: Japanese unexamined Patent Application Publication No. 2004-049225

Patent Document 2: Japanese unexamined Patent Application Publication No. 2010-081943

Patent Document 3: Japanese unexamined Patent Application Publication No. 2008-061608

Patent Document 4: Japanese Patent No. 3930897

Patent Document 5: Japanese unexamined Patent Application Publication No. 2008-068194

Patent Document 6: Japanese unexamined Patent Application Publication No. 2011-120538

Patent Document 7: Japanese unexamined Patent Application Publication No. 2006-006252

Patent Document 8: Japanese unexamined Patent Application Publication No. 2007-006745

Patent Document 9: Japanese unexamined Patent Application Publication No. 2012-139161

Patent Document 10: International Publication No. WO97/16198

Patent Document 11: Japanese unexamined Patent Application Publication (Translation of PCT Application) No. 2015-505851

Patent Document 12: Japanese unexamined Patent Application Publication (Translation of PCT Application) No. 2013-508416

Patent Document 13: Japanese unexamined Patent Application Publication No. 2015-091808

### SUMMARY OF THE INVENTION

### Object to be Solved by the Invention

An object of the present invention is to provide, when preparing food products for dysphagia patients, a food product for dysphagia patients having useful functions of probiotics and capable of delivering probiotics administered to a dysphagia patient to the intestines as viable cells by imparting functions of probiotics to such food products, and to provide a thickening composition therefor.

### Means to Solve the Object

For solving the above problems, the present inventors have conducted extensive studies on the production of a food product for dysphagia patients having the stability of probiotics against gastric juice decomposition action when a food product for dysphagia patients prepared using a probiotics-containing thickening composition for dysphagia patients is eaten, and found that when dried probiotic cells or a dried probiotic cell preparation and polysaccharides selected from polysaccharides consisting of xanthan gum or guar gum and hydrolysates of these polysaccharides are mixed to prepare a thickening composition as a powder mixture wherein the probiotics of the dried cells or the dried cell preparation and the polysaccharides are allowed to coexist in a contact state, the probiotics can be stably retained in the probiotics-containing thickening composition and also the probiotics prepared from the powder mixture in such a contact state have the stability against decomposition action of gastric juice, and also found that when a food product for dysphagia patients is produced using the thickening composition for dysphagia patients, the stability of probiotics in the thickening composition for dysphagia patients is assured and the stability of probiotics against juice decomposition action is also assured after the food product for dysphagia patients produced using the thickening composition is eaten thereby enabling a provision of a food product for dysphagia patients having useful functions of probiotics, whereby the present invention was accomplished.

More specifically, the thickening composition for dysphagia patients of the present invention consists of a thickening composition for dysphagia patients prepared as a powder mixture by mixing (A) dried probiotic cells or a dried probiotic cell preparation and (B) one or more polysaccharides selected from the group consisting of polysaccharides consisting of xanthan gum or guar gum and hydrolysates of these polysaccharides, wherein in the powder mixture, the probiotics of the dried cells or the dried cell preparation and the polysaccharides are allowed to coexist in a contact state. The thickening composition for dysphagia patients of the present invention is prepared as a powder mixture, and the probiotics in the thickening composition can be stably retained without employing special conditions such as refrigerated storage, and when a food product for dysphagia patients is produced using the thickening composition, a probiotics-containing food product for dysphagia patients having the stability against decomposition action of gastric juice after the food product is eaten can be provided. Further, in the present invention, the thickening composition is provided as a probiotics-containing thickening composition for producing food products for dysphagia patients and hence can be arranged even in the case of a food product for dysphagia patients mixed and prepared with various food product ingredients when eaten, and can also be applicable to the preparation of various food products for dysphagia patients at a site of eating, whereby a thickening composition arrangeable for the preparation of a broad range of probiotics-containing food products for dysphagia patients is provided, hence advantageous.

The thickening composition for dysphagia patients of the present invention can contain 0.01 parts by weight or more of (B) one or more polysaccharides selected from the group consisting of polysaccharides consisting of xanthan gum or guar gum and hydrolysates of these polysaccharides based on 1 part by weight of a dried cell weight of the probiotics of (A) dried probiotic cells or a dried probiotic cell preparation, and can preferably contain the polysaccharide of (B) in the range of 0.01 to 3000 parts by weight based on a dried cell weight of the probiotics of (A) of 1 part by weight. The polysaccharides of (B) to be mixed with the dried probiotic cells or the dried probiotic cell preparation of (A) preferably have an average particle size of 30 to 100 μm. Examples of the hydrolysate of a polysaccharide of (B) include a hydrolysate of a polysaccharide consisting of xanthan gum or guar gum obtained by acidically or enzymatically hydrolyzing the polysaccharide.

The thickening composition for dysphagia patients of the present invention can be prepared as a thickening composition for dysphagia patients by mixing (A) dried probiotic cells or a dried probiotic cell preparation and (B) polysaccharides consisting of xanthan gum or guar gum and polysaccharides consisting of hydrolysates of these polysaccharides, and further preparing a powder mixture wherein the probiotics of the dried cells or the dried cell preparation and the polysaccharides are allowed to coexist in a contact state, as a granule.

The present invention encompasses an invention of a method for producing a thickening composition for dysphagia patients, comprising employing a step, in a production step of a thickening composition for dysphagia patients, of mixing (A) dried probiotic cells or a dried probiotic cell preparation and (B) one or more polysaccharides selected from the group consisting of polysaccharides consisting of xanthan gum or guar gum and hydrolysates of these polysaccharides in a proportion of 0.01 parts by weight or more based on a dried cell weight of the probiotics of (A) dried probiotic cells or a dried probiotic cell preparation of 1 part by weight to prepare the thickening composition as a powder mixture wherein the probiotics of the dried cells or the dried cell preparation and the polysaccharides are allowed to coexist in a contact state. Further, the present invention encompasses an invention of the method for producing a thickening composition for dysphagia patients, further comprising subjecting to a granulation step the powder mixture of the dried probiotic cells or the dried probiotic cell preparation and the polysaccharides prepared in the powder mixture preparation step.

Further, the present invention encompasses an invention of a method for imparting to a probiotics-containing thickening composition for dysphagia patients stability of the probiotics in the thickening composition and stability of the probiotics against gastric juice decomposition action when a food product for dysphagia patients prepared using the thickening composition for dysphagia patients is eaten by mixing (A) dried probiotic cells or a dried probiotic cell preparation and (B) polysaccharides selected from polysaccharides consisting of xanthan gum or guar gum and hydrolysates of these polysaccharides to prepare the thickening composition as a powder mixture wherein the probiotics of the dried cells or the dried cell preparation and the polysaccharides are allowed to coexist in a contact state.

Furthermore, the present invention encompasses an invention of a method for imparting stability of the probiotics in a probiotics-containing thickening composition for dysphagia patients and stability against gastric juice decomposition action when a food product for dysphagia patients prepared using the thickening composition for dysphagia patients is eaten by further granulating the powder mixture wherein the dried probiotic cells or the dried probiotic cell preparation of (A) and the polysaccharides of (B) are allowed to coexist in a contact state.

Further, the present invention encompasses an invention of a method for producing a food product for dysphagia patients imparted with stability of the probiotics against gastric juice decomposition action after eaten comprising, in the production of a food product for dysphagia patients, producing by mixing the thickening composition for dysphagia patients of the present invention with food product ingredients for dysphagia patients.

More specifically, the present invention includes the following inventions:

[1] A thickening composition for dysphagia patients, wherein the thickening composition is prepared as a powder mixture by mixing (A) dried probiotic cells or a dried probiotic cell preparation and (B) one or more polysaccharides selected from the group consisting of polysaccharides consisting of xanthan gum or guar gum and hydrolysates of these polysaccharides, wherein in the powder mixture, the probiotics of the dried cells or the dried cell preparation and the polysaccharides are allowed to coexist in a contact state.

[2] The thickening composition for dysphagia patients according to [1], wherein the thickening composition contains 0.01 parts by weight or more of the (B) one or more polysaccharides selected from the group consisting of polysaccharides consisting of xanthan gum or guar gum and hydrolysates of these polysaccharides based on 1 part by weight of a dried cell weight of the probiotics of the (A) dried probiotic cells or dried probiotic cell preparation.

[3] The thickening composition for dysphagia patients according to [1] or [2], wherein the (B) one or more polysaccharides selected from the group consisting of polysaccharides consisting of xanthan gum or guar gum and hydrolysates of these polysaccharides to be mixed with the (A) dried probiotic cells or dried probiotic cell preparation have an average particle size of 30 to 100 μm.

[4] The thickening composition for dysphagia patients according to any one of [1] to [3], wherein the hydrolysate of a polysaccharide is a hydrolysate of a polysaccharide consisting of xanthan gum or guar gum obtained by acidically or enzymatically hydrolyzing the polysaccharide.

[5] The thickening composition for dysphagia patients according to any one of [1] to [4], wherein the (A) dried probiotic cells or dried probiotic cell preparation and the (B) one or more polysaccharides selected from the group consisting of polysaccharides consisting of xanthan gum or guar gum and hydrolysates of these polysaccharides are mixed and the powder mixture wherein the probiotics of the dried cells or the dried cell preparation and the polysaccharides are allowed to coexist in a contact state is further prepared as a granule.

[6] A method for producing a thickening composition for dysphagia patients, comprising employing a step, in a production step of a thickening composition for dysphagia patients, of mixing (A) dried probiotic cells or a dried probiotic cell preparation and (B) one or more polysaccharides selected from the group consisting of polysaccharides consisting of xanthan gum or guar gum and hydrolysates of these polysaccharides in a proportion of 0.01 parts by weight or more based on 1 part by weight of a dried cell weight of the probiotics of (A) dried probiotic cells or a dried probiotic cell preparation to prepare the thickening composition as a powder mixture wherein the probiotics of the dried cells or the dried cell preparation and the polysaccharides are allowed to coexist in a contact state.

[7] The method for producing a thickening composition for dysphagia patients according to [6], further comprising subjecting to a granulation step the powder mixture of the dried probiotic cells or the dried probiotic cell preparation and the polysaccharides prepared in the powder mixture preparation step.

[8] A method for imparting to a probiotics-containing thickening composition for dysphagia patients stability of the probiotics in the thickening composition and stability of the probiotics against gastric juice decomposition action when a food product for dysphagia patients prepared using the thickening composition for dysphagia patients is eaten by mixing (A) dried probiotic cells or a dried probiotic cell preparation and (B) one or more polysaccharides selected from the group consisting of polysaccharides consisting of xanthan gum or guar gum and hydrolysates of these polysaccharides to prepare the thickening composition as a powder mixture wherein the probiotics of the dried cells or the dried cell preparation and the polysaccharides are allowed to coexist in a contact state.

[9] The method for imparting to a probiotics-containing thickening composition for dysphagia patients stability of the probiotics in the thickening composition and stability of the probiotics against gastric juice decomposition action when a food product for dysphagia patients prepared using the thickening composition for dysphagia patients is eaten according to [8], further comprising granulating the powder mixture prepared by mixing the dried probiotic cells or the dried probiotic cell preparation and the polysaccharides.

[10] A method for producing a food product for dysphagia patients, having been imparted stability of the probiotics against gastric juice decomposition action after eaten, comprising producing the food product for dysphagia patients by mixing the thickening composition for dysphagia patients according to any one of [1] to [5] with a food product ingredient for dysphagia patients.

### Effect of the Invention

An object of the present invention is to provide, when preparing food products for dysphagia patients, a food product for dysphagia patients having useful functions of probiotics and capable of delivering probiotics administered to a dysphagia patient to the intestines as viable cells by imparting functions of probiotics to such food products. Additionally, the present invention provides a probiotics-containing thickening composition used for producing the food product for dysphagia patients. The probiotics-containing thickening composition for dysphagia patients of the present invention is prepared as a powder mixture, and the probiotics in the thickening composition can assure stability thereof without employing special conditions such as refrigerated storage, and further a food product for dysphagia patients produced using the thickening composition of the present invention has stability against decomposition action on the probiotics by gastric juice after the food product is eaten, whereby the thickening composition of the present invention can be used for the production of a stable probiotics-containing food product for dysphagia patients after eaten. Further, in the present invention, the thickening composition is provided as a probiotics-containing thickening composition for producing food products for dysphagia patients and hence can be applied even in the case of a food product for dysphagia patients mixed and prepared with various food product ingredients when eaten, whereby a thickening composition arrangeable for the production of a broad range of food products for dysphagia patients can be provided.

### MODE OF CARRYING OUT THE INVENTION

The thickening composition for dysphagia patients of the present invention is prepared as a powder mixture by mixing (A) dried probiotic cells or a dried probiotic cell preparation and (B) polysaccharides selected from the group consisting of polysaccharides consisting of xanthan gum or guar gum and hydrolysates of these polysaccharides, wherein in the powder mixture, the probiotics of the dried cells or the dried cell preparation and the polysaccharides are allowed to coexist in a contact state.

In the present invention, microorganisms used as probiotics in the thickening composition for dysphagia patients are defined as “living microorganisms which render beneficial actions to people by improving gut flora balance”, and examples of the probiotics include a microorganism belonging to the genus such as the Bifidobacterium genus, the Lactobacillus genus, the Enterococcus genus, or the Lactococcus genus.

Examples of the microorganism belonging to these genusa include the following microorganisms: microorganisms belonging to the Bifidobacterium genus include Bifidobacterium bifidum, Bifidobacterium longum, Bifidobacterium breve, Bifidobacterium infantis, Bifidobacterium adolescentis, and Bifidobacterium catenulatum; microorganisms belonging to the Lactobacillus genus include Lactobacillus acidophilus, Lactobacillus casei, Lactobacillus gasseri, Lactobacillus plantarum, Lactobacillus bulgaricus, Lactobacillus lactis, Lactobacillus delbrueckii, Lactobacillus fermentum, Lactobacillus helveticus, Lactobacillus paracasei, and Lactobacillus brevis; microorganisms belonging to the Enterococcus genus include Enterococcus faecalis and Enterococcus faecium; and microorganisms belonging to the Lactococcus genus include Lactococcus plantarum and Lactococcus raffinolactis. Probiotics in the present invention may be any of the above microorganisms, but the microorganisms categorized as the Lactobacillus genus, the Enterococcus genus, and the Lactococcus genus are more preferably used, and the microorganisms belonging to the genus Lactobacillus genus and the Enterococcus genus are further preferably used.

Probiotics used in the present invention may be, regardless of the original definition, dead cells but preferably cells in a proliferative state or cells transitionable to a proliferative state, that is, viable cells. For probiotics, a culture solution obtained by separating from and culturing wild probiotics in accordance with a known method may be used directly, or those prepared from such a culture solution may be used, or commercial probiotics may also be used. In the thickening composition for dysphagia patients of the present invention, probiotics are mixed as dried cells or a dried cell preparation, but dried cells are obtained by subjecting probiotic cells to drying treatment such as freeze drying. The dried cells can also be used as a dried cell preparation containing an excipient such as starch.

The thickening composition for dysphagia patients of the present invention is prepared as a powder mixture by mixing (A) dried probiotic cells or a dried probiotic cell preparation and (B) polysaccharides selected from the group consisting of polysaccharides consisting of xanthan gum or guar gum and hydrolysates of these polysaccharides, wherein in the powder mixture, they are allowed to coexist in a contact state. The one or more polysaccharides selected from the group consisting of polysaccharides consisting of xanthan gum or guar gum and hydrolysates of these polysaccharides may be used singly or used in combination. The polysaccharides are preferably in a spherical shape having a small particle size, and those having an average particle size of 30 to 100 μm are preferable and those having 50 to 100 μm are more preferable.

Hydrolysates of polysaccharides consisting of xanthan gum or guar gum can be prepared by hydrolyzing xanthan gum or guar gum using an acid or an enzyme. For the hydrolysate of xanthan gum or guar gum, either an acid hydrolysate or an enzyme hydrolysate may be used but as the thickening composition is for food products, a hydrolysate hydrolyzed using an enzyme is preferable. For the enzyme used for such a hydrolysis, examples of the suitable hydrolase can include galactomannanase, an enzyme derived from microorganisms of the Aspergillus genus and the Rhizopus genus for guar gum, and cellulase and xylanase derived from microorganisms for xanthan gum (see Japanese unexamined Patent Application Publication No. 02-248401, Japanese unexamined Patent Application Publication No. 05-117156, and Japanese unexamined Patent Application Publication No. 08-099884). Guar gum hydrolysates are commercially available such as “Sunfiber (manufactured by Taiyo Kagaku Co., Ltd.)” and “Fiberon (manufactured by DSP Gokyo Food & Chemical Co., Ltd.)” and thus easily accessible.

The thickening composition for dysphagia patients of the present invention uses, as the polysaccharide, polysaccharides selected from the group consisting of polysaccharides consisting of xanthan gum or guar gum and hydrolysates of these polysaccharides but, when preparing food products for dysphagia patients, can concurrently use a known method for facilitating dissolution and mixing in a sense of facilitating dissolution and mixing when producing food products for dysphagia patients using the thickening composition within a range in which the effect of the present invention is not interfered, for example, a range in which the survival of probiotics in an artificial gastric juice or gastric juice is not detrimentally affected. Examples of the method include a method for spraying a metal salt onto the powder surface of xanthan gum and drying to prevent the formation of lumps (Japanese Patent No. 3930897), a method for adding tara gum or glucomannan (Japanese unexamined Patent Application Publication No. 2011-254709), a method for adding readily soluble agar or readily soluble gelatin (Japanese unexamined Patent Application Publication No. 2005-245308), a method for spraying a potassium salt solution on the xanthan gum powder surface and fluid drying (Japanese unexamined Patent Application Publication No. 2010-81943), and a method for binding alginate and CMC onto the xanthan gum surface (Japanese unexamined Patent Application Publication No. 2008-61608).

The thickening composition for dysphagia patients of the present invention is produced as a powder mixture by mixing dried probiotic cells or a dried probiotic cell preparation and polysaccharides selected from the group consisting of polysaccharides consisting of xanthan gum or guar gum and hydrolysates of these polysaccharides, wherein in the powder mixture, the probiotics and the polysaccharides are allowed to coexist in a contact state, but can be produced by a method typically used as the production method of thickening composition for dysphagia patients with the only exception of including a step of allowing the probiotics and the polysaccharides to coexist in a contact state.

The method for allowing the probiotics and polysaccharides selected from the group consisting of polysaccharides consisting of xanthan gum or guar gum and hydrolysates of these polysaccharides to coexist in a contact state is not particularly limited, and examples include a method for mixing a powder of probiotics and the polysaccharides and a method for mixing and granulating probiotics and the polysaccharides. In the granulation method, it is preferable to mix probiotics and the polysaccharides in advance and then granulate the mixture.

The method for mixing a powder of probiotics and a powder of the polysaccharides may be any method using any apparatus as long as the powder of probiotics and the powder of the polysaccharides can be thoroughly mixed to be homogeneous.

The granulation method usable in the present invention is a known method, but spray granulation, vacuum freeze granulation, fluidized bed granulation, rolling granulation, and stirring granulation are preferably used. When using these methods, water and water containing a polysaccharide thickener can be used as a binder other than the raw materials described above. Granulation conditions may be suitably selected and determined depending on each of the granulation methods, but preferable conditions do not involve excessive heating or heat generation so as not to detrimentally affect the survival of probiotics, and it is preferable to use a method of granulating at a temperature lower than room temperature to avoid heat inactivation of the probiotics. When performing such a granulation, a granulation method can be employed in which a starch decomposition product containing a metal salt addable to food products such as a sodium salt, a potassium salt, a calcium salt, or a magnesium salt is contained to reduce lump formation (Japanese unexamined Patent Application Publication No. 2016-26507).

The amount of the probiotics contained in the thickening composition for dysphagia patients of the present invention may be suitably determined depending on the kind of probiotics to be administered and expected activity but the probiotics are typically contained, in term of viable cell count in 1 g of the composition of the present invention, in 1×10⁶ CFU or more, preferably 1×10⁷ CFU or more, and further preferably 1×10⁸ CFU or more.

The amount of the polysaccharides selected from the group consisting of polysaccharides consisting of xanthan gum or guar gum and hydrolysates of these polysaccharides to be allowed to coexist with the probiotics is, based on a dried cell weight of the probiotics of 1 part by weight, typically 0.01 parts by weight or more, preferably 0.02 parts by weight or more, more preferably 0.1 parts by weight or more, further preferably 0.2 parts by weight or more, particularly preferably 1 part by weight or more, and most preferably 10 parts by weight or more. There is no upper limit in the amount of the polysaccharides but the upper limit is, in consideration of the balance between an amount used as a texture modifier for the thickening composition for dysphagia patients of the present invention and a probiotics intake, 3000 parts by weight or less, preferably 2500 parts by weight or less, and more preferably 2000 parts by weight or less, based on a dried cell weight of the probiotics of 1 part by weight.

The thickening composition for dysphagia patients of the present invention may contain as necessary, other than the polysaccharides selected from the group consisting of polysaccharides consisting of xanthan gum or guar gum and hydrolysates of these polysaccharides, an excipient, a saccharide, a protein, a vitamin, a mineral, a colorant, and a flavor which are commonly used in thickening compositions for dysphagia patients as long as the survival of the probiotics is not detrimentally affected.

When preparing a food product for dysphagia patients, the thickening composition for dysphagia patients is used as dissolved or suspended in an aqueous medium such as water to prepare a liquid food product having a viscosity at which a subject (a person who eats), who is a dysphagia patient, can swallow, and then administered to the subject, and the administration method is preferably oral administration or tube administration by gastric fistula. The liquid food product administered by orally or gastric fistula is delivered to the intestines after passing through the intragastric environment, which is a harsh environment for the probiotics to survive. Intragastric pH is known to be typically 1 to 2 but increase to 4 to 5 immediately after meal and decrease again 2 to 3 hours after meal. For retention time in the stomach in the form of food, though depending on the kind of food, a liquid food product obtained by using the thickening composition of the present invention is estimated within 2 hours. Thus, when a survival rate is enhanced in the case of retaining such a food for about 1 to 2 hours, preferably 2 hours, in an artificial gastric juice imitating the intragastric environment by allowing the probiotics and polysaccharides selected from the group consisting of polysaccharides consisting of xanthan gum or guar gum and hydrolysates of these polysaccharides to coexist in a contact state in the thickening composition for dysphagia patients of the present invention, the method can be directly used as a method for enhancing the delivery rate of the probiotics administered to a dysphagia patient to the intestines as viable cells.

Examples of the artificial gastric juice in the present invention include an aqueous solution containing 0.25% by weight of a yeast extract, 0.5% by weight of peptone, 0.1% by weight of lactose, 0.1% by weight of polysorbate 80 (also called Tween80), 0.2% by weight of cysteine hydrochloride, and 0.2% by weight of sodium chloride, having pH of 2.7 to 4.0, and preferably pH of 2.8 to 3.5. Additionally, the retention time in an artificial gastric juice is 1 to 2 hours.

In the present invention, a viable cell remaining rate (survival rate) of the probiotics is determined as a percentage of the number of colonies (CFU) grown in an experimental plot treated with an artificial gastric juice to the number of colonies (CFU) grown in a control experimental plot treated with a diluted solution when a liquid in which the composition containing probiotics is dissolved or suspended in a diluted solution is added respectively to the diluted solution and an artificial gastric juice, mixed and suitably diluted with the diluted solution after a predetermined time has passed, subsequently poured to a medium in which the probiotics can be detected (for example, MRS medium) and cultured for to 3 days at a temperature (for example, 37° C.) and conditions (such as anaerobic conditions, as necessary) under which the probiotics can grow.

The effect of enhancing a survival rate of the probiotics in an artificial gastric juice can be achieved by allowing probiotics and polysaccharides selected from the group consisting of polysaccharides consisting of xanthan gum or guar gum and hydrolysates of these polysaccharides to coexist in a contact state, and particularly by mixing probiotics and xanthan gum and/or guar gum in a powder state, which is the method of the present invention.

The thickening composition for dysphagia patients of the present invention can be a liquid form to be administered to a dysphagia patient, and the aqueous medium for dissolving or suspending the composition usable to prepare such a liquid form include liquid nutrients usable in various soups, a miso soup, a green drink, and gastric fistula such as a milk, a milk-based drink, a lactic fermenting drink, a fruit juice-containing soft drink, a carbonated drink, a fruit juice, a vegetable drink, a tea drink, a sport drink, a functional drink, a vitamin supplement drink, a balanced nutrient supplement drink, a consomme clear soup, a potage soup, a cream soup, and a Chinese soup in addition to water and the diluted solutions described above as long as it can be administered to a dysphagia patent and has little affect to the survival of probiotics.

The amount used of the composition of the present invention when administering to a dysphagia patient varies depending on the swallowing function of a dysphagia patient to be administered and the kind of probiotics to be contained but is typically an amount in terms of a probiotics intake of, per single administration, 1×10⁷ CFU, preferably 1×10⁸ CFU, and more preferably 3×10⁸ CFU.

Hereinafter, the present invention is described in reference to examples but is not limited to these examples.

### EXAMPLES

### (1) Preparation of Thickening Compositions Containing Probiotics

Various thickening compositions were prepared using a commercial Enterococcus faecalis (hereinafter, also referred to as simply faecalis) preparation (containing cells in 6×10⁶ CFU or more/g and 95% by weight of starch), a xanthan gum preparation (xanthan gum 100%), a guar gum preparation (containing 70% guar gum), and dextrin (provided that comparative plots do not come under the thickening composition) in the formulation described in Table 1 to Table 3. In the tables, the addition amount of each component is shown as a part by weight when a dried cell weight in a cell preparation is 1 part by weight. Further, the starch as an excipient in the cell preparation and the dextrin in the guar gum preparation are also shown as adding components. The powder mixture products as the thickening compositions were prepared by weighing each of the powders and then mixing until the mixtures were homogeneous. Further, the granulated products were prepared by granulating a powder of each raw product using a fluidized bed granulator (manufactured by FREUND CORPORATION) under the conditions of an intake air temperature of 80° C., a granulation time of 10 minutes, a drying temperature of 80° C., and a drying time of 5 minutes.

### (2) Acid Resistance Test Using Gastric Juice

0.1 g each of the thickening compositions prepared in (1) was weighed and 10 ml of a diluted solution (containing 0.45% by weight of potassium dihydrogenphosphate, 0.6% by weight of disodium phosphate, 0.05% by weight of cysteine hydrochloride, 0.05% by weight of polysorbate 80 (Tween 80), and 0.1% by weight of agar) was added thereto and mixed thoroughly. The mixture was further diluted 100-fold using the diluted solution and used as a stock solution for the test. On the other hand, an aqueous solution containing 0.25% by weight of a yeast extract, 0.5% by weight of peptone, 0.1% by weight of lactose, 0.1% by weight of polysorbate 80 (Tween 80), 0.2% by weight of cysteine hydrochloride, and 0.2% by weight of sodium chloride was prepared and adjusted to pH 3.5 and used as an artificial gastric juice.

Considering the cell count in the faecalis preparation used, the diluted solution was added to the stock solution for the test to dilute in such a way that a cell count was 12,000 cells/ml, the artificial gastric juice was further added to dilute in such a way that the cell count was 120 cells/ml and the mixture was used as an artificial gastric juice treated plot. The solution of the artificial gastric juice treated plot was partially moved immediately after prepared to a 37° C. thermostat chamber together with the container, allowed to stand for 60 minutes or 120 minutes and used as an artificial gastric juice treated plot 60 minutes and an artificial gastric juice treated plot 120 minutes, respectively.

1 ml was collected from the remaining solution of the artificial gastric juice treated plot, smeared to a petri dish, subsequently 15 ml of 4% salt-containing MRS agar medium (medium in which sodium chloride is added in such a way as to be 4% by weight and agar is further added thereto in such a way as to be 1.5% by weight to MRS medium manufactured by Becton, Dickinson and Company Japan) was added and solidified in the petri dish. The petri dish was incubated at 37° C. under an aerobic condition for 48 hours (artificial gastric juice treated plot 0 minutes). Additionally, the same procedure was also performed for the experimental plots treated with the artificial gastric juice treated plot for 60 minutes or 120 minutes.

After cultured, the number of colonies grown in the agar medium in the petri dish was counted. When CFU of the artificial gastric juice treated plot 0 minutes was 100, CFUs of the artificial gastric juice treated plot 60 minutes and the artificial gastric juice treated plot 120 minutes were calculated as survival rates of after treated with the artificial gastric juice. The results are shown sequentially in Table 1 to Table 4. Additionally, in the tables, a survival rate (fold) in each of the experimental plots, when the survival rate of the comparative plot was 1, was shown in the parentheses.

TABLE 1
Probiotics-containing thickening compositions and
cell survival rates in artificial gastric juice (1)

| | | | | | Survival rate in artificial | |
|---|---|---|---|---|---|---|
| Experimental | | | Part by | | gastric juice treated plots (%) | |
| plot | Components | | weight | Form | 60 Minutes | 120 Minutes |
| 1 | Cell | Cell | 1 | Powder | 76.3 | 18.2 |
| (Comparative | preparation | Starch | 19 | | (1 time) | (1 time) |
| plot) | Others | | 0 | | | |
| 2 | Cell | Cell | 1 | Powder | 82.3 | 50.2* |
| | preparation | Starch | 19 | | (1.1 times) | (2.8 times) |
| | Xanthan gum | | 1980 | | | |
| 3 | Cell | Cell | 1 | Powder | 84.1 | 46.2* |
| | preparation | Starch | 19 | | (1.1 times) | (2.5 times) |

| | |
|---|---|
| Guar gum | 1386 |
| Dextrin | 594 |

n = 3
*There is a significant difference to the survival rate in experimental plot 1 (comparative plot) at a risk factor of 5% (P < 0.05)

As shown in Table 1, the thickening compositions obtained by adding xanthan gum (experimental plot 2: 1980 parts by weight per part by weight of cells) or guar gum (experimental plot 3: 1386 parts by weight per part by weight of cells) to and mixing with cells of faecalis had high cell survival rates even after the thickening composition was dissolved in the artificial gastric juice for 60 minutes or 120 minutes. Particularly, there were significant differences in the cell survival rate 120 minutes after dissolution.

The guar gum preparation used herein contains dextrin as the excipient and hence the possible contribution of this dextrin to the enhancement in the survival rate in experimental plot 3 cannot be ruled out, however, the enhancement in the survival rate is similarly confirmed with a significant difference in dextrin-free experimental plot 2 whereby the effect in experimental plot 3 is estimated to have been rendered by guar gum.

The cell protective effects by xanthan gum and guar gum in the artificial gastric juice were confirmed, and hence subsequent tests for confirming the ratio of a xanthan gum amount to a cell amount and the cell protective effect was performed. The results are shown in Table 2.

TABLE 2
Probiotics-containing thickening compositions and
cell survival rates in artificial gastric juice (2)

| | | | | | Survival rate in artificial | |
|---|---|---|---|---|---|---|
| Experimental | | | Part by | | gastric juice treated plots (%) | |
| plot | Components | | weight | Form | 60 Minutes | 120 Minutes |
| 1 | Cell | Cell | 1 | Powder | 58.2 | 10.9 |
| (Comparative | preparation | Starch | 19 | | (1 time) | (1 time) |
| plot) | Others | | 0 | | | |
| 2 | Cell | Cell | 1 | Powder | 78.3 | 33.7* |
| | preparation | Starch | 19 | | (1.3 times) | (3.1 times) |
| | Xanthan gum | | 1980 | | | |
| 4 | Cell | Cell | 1 | Powder | 74.7 | 32.0* |
| | preparation | Starch | 19 | | (1.3 times) | (2.9 times) |
| | Xanthan gum | | 46.7 | | | |
| 5 | Cell | Cell | 1 | Powder | 63.0 | 27.6* |
| | preparation | Starch | 19 | | (1.1 times) | (2.5 times) |

| | |
|---|---|
| Xanthan gum | 2.2 |

n = 3
*There is a significant difference to the survival rate in experimental plot 1 (comparative plot) at a risk factor of 5% (P < 0.05)

As shown in Table 2, the thickening compositions obtained by adding xanthan gum to and mixing with cells of faecalis had high cell survival rates even after each of the compositions was dissolved in the artificial gastric juice for 60 minutes or 120 minutes. As such an effect did not decrease notably even in the experimental plots in which ratios of xanthan gum to cells were decreased (experimental plots 4 and 5), the same test was separately performed by further decreasing a ratio of xanthan gum. The results are shown in Table 3.

TABLE 3
Probiotics-containing thickening compositions and
cell survival rates in artificial gastric juice (3)

| | | | | | Survival rate in artificial | |
|---|---|---|---|---|---|---|
| Experimental | | | Part by | | gastric juice treated plots (%) | |
| plot | Components | | weight | Form | 60 Minutes | 120 Minutes |
| 1 | Cell | Cell | 1 | Powder | 41.4 | 5.3 |
| (Comparative | preparation | Starch | 19 | | (1 time) | (1 time) |
| plot) | Others | | 0 | | | |
| 6 | Cell | Cell | 1 | Powder | 60.7* | 25.6* |
| | preparation | Starch | 19 | | (1.5 times) | (4.8 times) |
| | Xanthan gum | | 0.2 | | | |
| 7 | Cell | Cell | 1 | Powder | 65.5* | 16.5* |
| | preparation | Starch | 19 | | (1.6 times) | (3.1 times) |

| | |
|---|---|
| Xanthan gum | 0.02 |

n = 3

As shown in Table 3, the cell protective effect was also confirmed with significant differences in the experimental plots in which the ratios of xanthan gum to cells were further decreased (experimental plots 6 and 7) compared to experimental plot 5.

The tests shown in Table 1 to Table 3 all used the powder mixtures of the cells and the thickeners, and hence tests were performed for confirming the cell protective effect by xanthan gum or guar gum in the artificial gastric juice when the composition is a granule. The results are shown in Table 4.

TABLE 4
Probiotics-containing thickening compositions and
cell survival rates in artificial gastric juice (4)

| | | | | | Survival rate in artificial | |
|---|---|---|---|---|---|---|
| Experimental | | | Part by | | gastric juice treated plots (%) | |
| plot | Components | | weight | Form | 60 Minutes | 120 Minutes |
| 1 | Cell | Cell | 1 | Powder | 60.0 | 21.2 |
| (Comparative | preparation | Starch | 19 | | (1 time) | (1 time) |
| plot) | Others | | 0 | | | |
| 8 | Cell | Cell | 1 | Powder | 94.1* | 88.8* |
| | preparation | Starch | 19 | | (1.6 times) | (4.2 times) |
| | Xanthan gum | | 600 | | | |
| | Guar gum | | 1.4 | | | |
| | Dextrin | | 1379 | | | |
| 9 | Cell | Cell | 1 | Granule | 90.6* | 79.3* |
| | preparation | Starch | 19 | | (1.5 times) | (3.7 times) |

| | |
|---|---|
| Xanthan gum | 600 |
| Guar gum | 1.4 |
| Dextrin | 1379 |

n = 3
*There is a significant difference to the survival rate in experimental plot 1 (comparative plot) at a risk factor of 5% (P < 0.05)

As shown in Table 4, the cell protective effect by xanthan gum or guar gum in the artificial gastric juice was similarly confirmed not only in the case of the powder mixtures but also in the case of the granules. It is generally concerned that the cells are damaged by the granulation procedure and the survival rate is detrimentally affected, however, the cell survival rates little affected when xanthan gum or guar gum was allowed to coexist with cells in a contact state verify that the thickening composition for dysphagia patients of the present invention can also be a granule.

### (3) Cell Protective Effects by Xanthan Gum, Guar Gum, and Guar Gum Hydrolysate in the Artificial Gastric Juice (Additional Test using Enterococcus faecalis)

When the above test was performed again by changing the lot of faecalis, a rather high survival rate was confirmed even in a comparative plot (an experimental plot to which xanthan gum or guar gum was not added), and hence tests were performed by the same procedure by decreasing pH of the artificial gastric juice to 3.2 (pH was 3.5 in the above tests). Further, an effect of a guar gum hydrolysate (product name: Sunfiber, manufactured by Taiyo Kagaku Co., Ltd.) as a cell protective component was also investigated in addition to xanthan gum and guar gum. Amounts of xanthan gum, guar gum, and the guar gum hydrolysate were adjusted in such a way as to be 99 parts by weight per part by weight of the cells. The results are shown in Table 5.

TABLE 5
Probiotics-containing thickening compositions and
cell survival rates in artificial gastric juice (5)

| | | | | | Survival rate in artificial | |
|---|---|---|---|---|---|---|
| Experimental | | | Part by | | gastric juice treated plots (%) | |
| plot | Components | | weight | Form | 60 Minutes | 120 Minutes |
| 10 | Cell | Cell | 1 | Powder | 1.8 | 0 |
| (Comparative | preparation | Starch | 19 | | (1 time) | |
| plot) | Others | | 0 | | | |
| 11 | Cell | Cell | 1 | Powder | 7.2* | 0.7* |
| | preparation | Starch | 19 | | (4.0 times) | (∞) |
| | Xanthan gum | | 1980 | | | |
| 12 | Cell | Cell | 1 | Powder | 0.2 | 0 |
| | preparation | Starch | 19 | | (0.1 times) | (0 times) |
| | Guar gum | | 1386 | | | |
| | Dextrin | | 594 | | | |
| 13 | Cell | Cell | 1 | Powder | 3.4 | 0.8* |
| | preparation | Starch | 19 | | (1.9 times) | (∞) |

| | |
|---|---|
| Guar gum hydrolysate | 1980 |

n = 3
*There is a significant difference to the survival rate in experimental plot 10 (comparative plot) at a risk factor of 5% (P < 0.05)

As shown in Table 5, the tests were performed by decreasing pH of the artificial gastric juice to 3.2, the survival rates were apparently decreased compared to the tests at pH 3.5 but the cell protective effect was confirmed to have remained in xanthan gum and the guar gum hydrolysate.

### (4) Cell Protective Effect by Xanthan Gum in the Artificial Gastric Juice (Tests using Lactobacillus acidophilus)

The same tests were performed using a Lactobacillus acidophilus (hereinafter also referred to as simply acidophilus) preparation in place of the Enterococcus faecalis preparation, which was the test cell in the above tests, and xanthan gum as the polysaccharide.

The acidophilus preparation used herein contained both cells of acidophilus and faecalis, and hence the viable cell count of acidophilus for measurement of survival rates was estimated by using MRS agar medium in which both acidophilus and faecalis can grow and MRS agar medium to which 4% sodium chloride was added in which only faecalis can grow and calculating a difference.

Additionally, pH of the artificial gastric juice for investigating the acidophilus cell protective effect by xanthan gum was adjusted to 2.7 and 2.8. The results are shown in Table 6.

TABLE 6
Probiotics-containing thickening compositions and cell survival rates in artificial gastric juice (6)

| | | | | | | Survival rate in artificial | |
|---|---|---|---|---|---|---|---|
| | pH of | | | | | gastric juice treated plots (%) | |
| Experimental | artificial | | | Part by | | 60 | 120 |
| plot | gastric juice | Components | | weight | Form | Minutes | Minutes |
| 14 | 2.7 | Cell | Cell | 1 | Powder | 0.5 | 0 |
| (Comparative | | preparation | Starch | 19 | | | |
| plot) | | Others | | 0 | | | |
| 15 | | Cell | Cell | 1 | Powder | 0.8* | 0 |
| | | preparation | Starch | 19 | | | |
| | | Xanthan gum | | 1980 | | | |
| 16 | 2.8 | Cell | Cell | 1 | Powder | 2.1 | 0.2 |
| (Comparative | | preparation | Starch | 19 | | | |
| plot) | | Others | | 0 | | | |
| 17 | | Cell | Cell | 1 | Powder | 4.7* | 0.7* |
| | | preparation | Starch | 19 | | | |

| | |
|---|---|
| Xanthan gum | 1980 |

n = 3
*There are significant differences to the survival rate in experimental plot 14 (comparative plot) and experimental plot 16 (comparative plot) at a risk factor of 5% (P < 0.05)

As shown in Table 6, the cell protective effects by xanthan gum in the artificial gastric juice at pH 2.7 and 2.8 were similarly confirmed even in the tests in which the test cell was changed from faecalis to acidophilus.

### INDUSTRIAL APPLICABILITY

An object of the present invention is to provide, when preparing food products for dysphagia patients, a food product for dysphagia patients having useful functions of probiotics and capable of delivering probiotics administered to a dysphagia patient to the intestines as viable cells by imparting functions of probiotics to such food products. Additionally, the present invention provides a probiotics-containing thickening composition used for producing the food product for dysphagia patients. The probiotics-containing thickening composition for dysphagia patients of the present invention is prepared as a powder mixture, and the probiotics in the thickening composition can assure stability thereof without employing special conditions such as refrigerated storage, and further a food product for dysphagia patients produced using the thickening composition of the present invention has stability against decomposition action on the probiotics by gastric juice after the food product is eaten, whereby the thickening composition of the present invention can be used for the production of a stable probiotics-containing food product for dysphagia patients after eaten. Further, in the present invention, the thickening composition is provided as a probiotics-containing thickening composition for producing food products for dysphagia patients and hence can be applied even in the case of a food product for dysphagia patients mixed and prepared with various food product ingredients when eaten.

1. A thickening composition for dysphagia patients, wherein the thickening composition is prepared as a powder mixture by mixing (A) dried probiotic cells or a dried probiotic cell preparation and (B) one or more polysaccharides selected from the group consisting of polysaccharides consisting of xanthan gum or guar gum and hydrolysates of these polysaccharides, wherein in the powder mixture, the probiotics of the dried cells or the dried cell preparation and the polysaccharides are allowed to coexist in a contact state.

2. The thickening composition for dysphagia patients according to claim 1, wherein the thickening composition contains 0.01 parts by weight or more of the (B) one or more polysaccharides selected from the group consisting of polysaccharides consisting of xanthan gum or guar gum and hydrolysates of these polysaccharides based on 1 part by weight of a dried cell weight of the probiotics of the (A) dried probiotic cells or dried probiotic cell preparation.

3. The thickening composition for dysphagia patients according to claim 1, wherein the (B) one or more polysaccharides selected from the group consisting of polysaccharides consisting of xanthan gum or guar gum and hydrolysates of these polysaccharides to be mixed with the (A) dried probiotic cells or dried probiotic cell preparation have an average particle size of 30 to 100 μm.

4. The thickening composition for dysphagia patients according to claim 1, wherein the hydrolysate of a polysaccharide is a hydrolysate of a polysaccharide consisting of xanthan gum or guar gum obtained by acidically or enzymatically hydrolyzing the polysaccharide.

5. The thickening composition for dysphagia patients according to claim 1, wherein the (A) dried probiotic cells or dried probiotic cell preparation and the (B) one or more polysaccharides selected from the group consisting of polysaccharides consisting of xanthan gum or guar gum and hydrolysates of these polysaccharides are mixed and the powder mixture wherein the probiotics of the dried cells or the dried cell preparation and the polysaccharides are allowed to coexist in a contact state is further prepared as a granule.

6. A method for producing a thickening composition for dysphagia patients, comprising employing a step, in a production step of a thickening composition for dysphagia patients, of mixing (A) dried probiotic cells or a dried probiotic cell preparation and (B) one or more polysaccharides selected from the group consisting of polysaccharides consisting of xanthan gum or guar gum and hydrolysates of these polysaccharides in a proportion of 0.01 parts by weight or more based on 1 part by weight of a dried cell weight of the probiotics of (A) dried probiotic cells or a dried probiotic cell preparation to prepare the thickening composition as a powder mixture wherein the probiotics of the dried cells or the dried cell preparation and the polysaccharides are allowed to coexist in a contact state.

7. The method for producing a thickening composition for dysphagia patients according to claim 6, further comprising subjecting to a granulation step the powder mixture of the dried probiotic cells or the dried probiotic cell preparation and the polysaccharides prepared in the powder mixture preparation step.

8. A method for imparting to a probiotics-containing thickening composition for dysphagia patients stability of the probiotics in the thickening composition and stability of the probiotics against gastric juice decomposition action when a food product for dysphagia patients prepared using the thickening composition for dysphagia patients is eaten, by mixing (A) dried probiotic cells or a dried probiotic cell preparation and (B) one or more polysaccharides selected from the group consisting of polysaccharides consisting of xanthan gum or guar gum and hydrolysates of these polysaccharides to prepare the thickening composition as a powder mixture wherein the probiotics of the dried cells or the dried cell preparation and the polysaccharides are allowed to coexist in a contact state.

9. The method for imparting to a probiotics-containing thickening composition for dysphagia patients stability of the probiotics in the thickening composition and stability of the probiotics against gastric juice decomposition action when a food product for dysphagia patients prepared using the thickening composition for dysphagia patients is eaten according to claim 8, further comprising granulating the powder mixture prepared by mixing the dried probiotic cells or the dried probiotic cell preparation and the polysaccharides.

10. A method for producing a food product for dysphagia patients, having been imparted stability of the probiotics against gastric juice decomposition action after eaten, comprising producing the food product for dysphagia patients by mixing the thickening composition for dysphagia patients according to claim 1 with a food product ingredient for dysphagia patients.

11. The thickening composition for dysphagia patients according to claim 2, wherein the (B) one or more polysaccharides selected from the group consisting of polysaccharides consisting of xanthan gum or guar gum and hydrolysates of these polysaccharides to be mixed with the (A) dried probiotic cells or dried probiotic cell preparation have an average particle size of 30 to 100 μm.

12. The thickening composition for dysphagia patients according to claim 2, wherein the hydrolysate of a polysaccharide is a hydrolysate of a polysaccharide consisting of xanthan gum or guar gum obtained by acidically or enzymatically hydrolyzing the polysaccharide.

13. The thickening composition for dysphagia patients according to claim 3, wherein the hydrolysate of a polysaccharide is a hydrolysate of a polysaccharide consisting of xanthan gum or guar gum obtained by acidically or enzymatically hydrolyzing the polysaccharide.

14. The thickening composition for dysphagia patients according to claim 11, wherein the hydrolysate of a polysaccharide is a hydrolysate of a polysaccharide consisting of xanthan gum or guar gum obtained by acidically or enzymatically hydrolyzing the polysaccharide.

15. The thickening composition for dysphagia patients according to claim 2, wherein the (A) dried probiotic cells or dried probiotic cell preparation and the (B) one or more polysaccharides selected from the group consisting of polysaccharides consisting of xanthan gum or guar gum and hydrolysates of these polysaccharides are mixed and the powder mixture wherein the probiotics of the dried cells or the dried cell preparation and the polysaccharides are allowed to coexist in a contact state is further prepared as a granule.

16. The thickening composition for dysphagia patients according to claim 3, wherein the (A) dried probiotic cells or dried probiotic cell preparation and the (B) one or more polysaccharides selected from the group consisting of polysaccharides consisting of xanthan gum or guar gum and hydrolysates of these polysaccharides are mixed and the powder mixture wherein the probiotics of the dried cells or the dried cell preparation and the polysaccharides are allowed to coexist in a contact state is further prepared as a granule.

17. The thickening composition for dysphagia patients according to claim 4, wherein the (A) dried probiotic cells or dried probiotic cell preparation and the (B) one or more polysaccharides selected from the group consisting of polysaccharides consisting of xanthan gum or guar gum and hydrolysates of these polysaccharides are mixed and the powder mixture wherein the probiotics of the dried cells or the dried cell preparation and the polysaccharides are allowed to coexist in a contact state is further prepared as a granule.

18. The thickening composition for dysphagia patients according to claim 11, wherein the (A) dried probiotic cells or dried probiotic cell preparation and the (B) one or more polysaccharides selected from the group consisting of polysaccharides consisting of xanthan gum or guar gum and hydrolysates of these polysaccharides are mixed and the powder mixture wherein the probiotics of the dried cells or the dried cell preparation and the polysaccharides are allowed to coexist in a contact state is further prepared as a granule.

19. The thickening composition for dysphagia patients according to claim 12, wherein the (A) dried probiotic cells or dried probiotic cell preparation and the (B) one or more polysaccharides selected from the group consisting of polysaccharides consisting of xanthan gum or guar gum and hydrolysates of these polysaccharides are mixed and the powder mixture wherein the probiotics of the dried cells or the dried cell preparation and the polysaccharides are allowed to coexist in a contact state is further prepared as a granule.

20. The thickening composition for dysphagia patients according to claim 13, wherein the (A) dried probiotic cells or dried probiotic cell preparation and the (B) one or more polysaccharides selected from the group consisting of polysaccharides consisting of xanthan gum or guar gum and hydrolysates of these polysaccharides are mixed and the powder mixture wherein the probiotics of the dried cells or the dried cell preparation and the polysaccharides are allowed to coexist in a contact state is further prepared as a granule.
